(19) United States
(12) Patent Application Publication
(10) Pub. No.: US 2016/0175257 A1
(43) Pub. Date: Jun. 23, 2016

(54) SOLID ORAL DOSAGE FORM OF METHYLPHENIDATE OR SALTS THEREOF

(71) Applicant: GAVIS PHARMACEUTICALS, Somerset, NJ (US)

(72) Inventors: Prabagaran Chandrasekaran, Somerset, NJ (US); James Garegnani, Somerset, NJ (US)

(73) Assignee: GAVIS PHARMACEUTICALS, Somerset, NJ (US)

(21) Appl. No.: 14/577,962

(22) Filed: Dec. 19, 2014

(51) Int. Cl. A61K 9/28; A61K 31/4458

(52) U.S. Cl. CPC A61K 9/2886; A61K 31/4458; A61K 9/28; A61K 9/2893; A61K 9/2866

(57) ABSTRACT

A solid oral dosage form of methylphenidate or salts thereof which provide a rapid initial onset of action and a prolonged duration of effect is provided. The solid oral dosage form comprises a matrix core containing methylphenidate or salts thereof coated by a modified release coating and an outer methylphenidate immediate release coating.

### BACKGROUND OF THE INVENTION

(a) Field of the Invention

The present invention is directed to a solid oral dosage form of methylphenidate or salts thereof which provides a rapid initial onset of action and a prolonged duration of effect. The solid oral dosage form comprises a matrix core containing methylphenidate or salts thereof coated by a modified release coating and an outer methylphenidate immediate release coating. The invention is further directed to the use of said oral dosage forms for treating a condition susceptible to treatment with methylphenidate. Additionally, the present invention provides a method of manufacture of said dosage forms.

(b) Description of the Related Art

Methylphenidate is a central nervous system stimulant used to treat Attention-Deficit Disorder (ADD) and Attention-Deficit Hyperactivity Disorder (ADHD), available commercially as, e.g. RITALIN SR™, CONCERTA™, METADATE™ CD capsules and METEADATE™ ER Tablets. The beneficial results seen by clinicians in the treatment of ADD and ADHD has resulted in widespread use of methylphenidate in more than two million patients annually. Methylphenidate exists in four separate optical isomers, 1-threo, d-erythro, d-threo and 1-erythro, with the threo pair of enantiomers (racemate) of methylphenidate hydrochloride generally administered for the treatment of ADD and ADHD. Methylphenidate hydrochloride is a mild central nervous system stimulant with pharmacological properties similar to that of amphetamines. The side effects which are associated with the use of methylphenidate include anorexia, weight loss, insomnia, dizziness and dysphoria. Methylphenidate is categorized by the DEA as a Schedule II controlled substance because it produces a euphoric effect when administered intravenously or through inhalation or ingestion, and thus carries a high potential for abuse.

Numerous sustained release formulations of methylphenidate which provide for a slow release of the medication over a predetermined period of time have been developed and some of these formulations are described in U.S. Pat. Nos. 6,340,476; 6,344,215; 6,372,254; 6,368,626; 5,837,284 and 6,034,101, incorporated herein by reference. However, when methylphenidate is delivered at a steady state over a sustained period of time, acute tolerance often develops. The undesirable clinical effects of steady state sustained release formulations are overcome by multiple dosing whereby the intensity of the therapeutic effects of the methylphenidate can be maintained.

U.S. Pat. Nos. 6,919,337 and 6,930,129 disclose a dosage form and a method of administering methylphenidate in a controlled release formulation providing an ascending release rate. The dosage form disclosed in the '337 and '129 patents comprises a layered tablet core including an expanding “push layer” which serves to facilitate release of the active ingredient contained within the tablet.

U.S. Pat. No. 6,034,101 discloses a sustained release tablet form of methylphenidate hydrochloride that provides an ascending dose of the drug for regulation of its tolerance. Controlled release of the medication is achieved by a hydrogel matrix comprising a hydrophilic polymer.

U.S. Pat. Nos. 7,083,808 and 7,438,930 disclose a modified/controlled release methylphenidate formulation which provides a rapid initial onset of effect and a prolonged duration of effect. The formulation comprises a controlled release matrix of methylphenidate coated with a hydrophobic material followed by an enteric coating.

U.S. Pat. No. 8,709,477 discloses a composition comprising coated beads or pellets containing a methylphenidate layer, a sustained release coating and a controlled release enteric coating. The coated beads or pellets form cores of the extended release tablet which is further coated with an outer immediate release layer of methylphenidate.

U.S. Pat. Application Pub. No. 2009/0110728 discloses a modified release dosage form of methylphenidate that exhibits a zero order release. The dosage form comprises a matrix core of methylphenidate and a hydrophobic material.

The formulations of methylphenidate disclosed in the prior art employs either a matrix system, a modified or controlled release coating system or a combination of both to provide modified release of methylphenidate from the dosage form. However, the formulations employing both a matrix and a modified or controlled release coating system often falls short on providing a desired drug release from the dosage form or exhibits a non-reproducible drug release pattern. The release pattern of methylphenidate from the known modified release formulations may vary from dosage form to dosage form. This is evident by a recent suspension of therapeutic equivalence rating (or ‘AB’ rating) issued by US-FDA for a commercialized methylphenidate extended release tablet product in the United States.

Methylphenidate formulations of the prior art also require complicated manufacturing processes, commercial manufacturing of which in turn consume significant amounts of time and are cost intensive.

There remains a need for a novel methylphenidate modified release formulation which not only exhibits prolonged duration of effect by extending release of methylphenidate from the formulation but also possess batch-to-batch reproducibility of the release profile. The manufacturing process for such a formulation also needs to be robust for production on a commercial scale.

### SUMMARY OF THE INVENTION

The present invention provides solid oral dosage forms of methylphenidate. The dosage form comprise a matrix core comprising methylphenidate or salts thereof, a hydrophobic material and a gel forming hydrophilic material. The matrix core is further coated with a modified release coating to further control the release of methylphenidate from the matrix core. An outer coating comprising methylphenidate or salts thereof is provided over the modified release coating. Also provided is the solid oral dosage form of methylphenidate which is stable at room temperature. Methods of treating patients in need thereof with such methylphenidate dosage form are further provided by the invention.

In one aspect, the invention provides a solid oral dosage form comprising:
(a) a matrix core which comprises methylphenidate or a salt thereof, at least one hydrophobic material, at least one gel forming hydrophilic material and one or more pharmaceutically acceptable excipients,
(b) a modified release coating surrounding the matrix core, which comprises at least one pH-independent polymer,
(c) optionally, a pH dependent release modifying coating surrounding the modified release coating,
(d) optionally, a seal coating surrounding the pH dependent release modifying coating, and
(e) a coating of methylphenidate or a salt thereof surrounding the modified release coating, pH dependent release modifying coating, or seal coating.

The modified release coating comprises up to about 10% w/w of the hydrophobic material and up to about 22% w/w of the gel forming hydrophilic material by total weight of the uncoated matrix core.

In another aspect, the modified release coating comprises at least one pH-independent polymer dissolved or dispersed in hydro-alcoholic solvent mixture. In an embodiment, the modified release coating is devoid of a pH-dependent polymer or substance. In another embodiment, the modified release coating is applied over the matrix core without any intermediate barrier coating.

In another aspect, the coating of methylphenidate or a salt thereof exhibits immediate release. In an embodiment, the outer coating comprises up to about 20% of methylphenidate or a salt thereof by total amount of methylphenidate in the dosage form.

In another aspect, the hydrophobic material in the matrix core is not a polymeric material.

In another aspect, the pH dependent release modifying coating comprises at least one pH-dependent polymer, at least one pore hydrophilic pore forming agent and at least one plasticizer.

In another aspect, the invention provides a solid oral dosage form exhibiting an in-vitro dissolution as follows:
(a) not less than 10% methylphenidate is dissolved after 1 hour;
(b) not less than 40% methylphenidate is dissolved after 4 hours;
(c) not less than 80% methylphenidate is dissolved after 8 hours, and
(d) not less than 90% methylphenidate is dissolved after 12 hours.

In another aspect, the invention provides a solid oral dosage form of methylphenidate or a salt thereof having a release profile that is comparable to the dosage form of methylphenidate or a salt thereof subjected to stability testing at 40° C. and 75% relative humidity for period of 6 months.

In another aspect, the invention provides a method of manufacturing the dosage form of methylphenidate or a salt thereof, which process comprises the steps of:
(a) forming a matrix core by compressing methylphenidate or a salt thereof, at least one hydrophobic material, at least one gel forming hydrophilic material,
(b) applying the modified release coating over the matrix core,
(c) optionally, applying the pH dependent release modifying coating over the modified release coating,
(d) optionally, applying the seal coating over the modified release coating, and
(e) applying the coating of methylphenidate or a salt thereof over the modified release coating, pH dependent release modifying coating, or seal coating.

In another aspect, the invention provides a methylphenidate modified release tablet comprising:
(a) a matrix core which comprises methylphenidate or a salt thereof, at least one hydrophobic material, at least one gel forming hydrophilic material and one or more pharmaceutically acceptable excipients,
(b) a modified release coating surrounding the matrix core, wherein the modified release coating consists of at least one pH-independent polymer,
(c) optionally, a seal coating surrounding the pH dependent release modifying coating, and
(d) an outer immediate release coating comprises up to about 20% of methylphenidate or a salt thereof by total amount of methylphenidate in the dosage form;

wherein the modified release coating consisting of up to about 10% w/w of the hydrophobic material and up to about 22% w/w of the gel forming hydrophilic material by total weight of the uncoated matrix core.

In another aspect, the invention provides a method for treating a patient having a condition amenable to treatment with methylphenidate, the method comprising administering to the patient the dosage form as substantially described herein.

In another aspect, the invention provides a method for treating Attention Deficit Disorder or Attention Deficit/Hyperactivity Disorder in a patient in need thereof, said method comprising: providing a dosage form as substantially described herein and administering said dosage form to the patient.

Still other aspects and advantages of the invention will be apparent from the following detailed description of the invention.

### BRIEF DESCRIPTION OF THE DRAWINGS

FIG. 1 represents a comparison of the dissolution profile of Concerts® ER Tablet and Formulations 2 and 3 of Example 1.

FIG. 2 represents a comparison of the dissolution profile of Concerts® ER Tablet and Formulations 4 and 5 of Example 1.

FIG. 3 represents a comparison of the dissolution profile of Formulation 2 of Example 1 and that of same Formulation 2 after being subjected to a stability study over 6 months.

FIG. 4 represents a comparison of the dissolution profile of Concerts® ER Tablet and Formulations 1 and 2 of Comparative Example 1.

FIG. 5 represents a comparison of the dissolution profile of Concerts® ER Tablet and Formulations 3 and 4 of Comparative Example 1.

FIG. 6 represents a comparison of the dissolution profile of Concerts® ER Tablet and Formulations 5 and 6 of Comparative Example 1.

### DETAILED DESCRIPTION OF THE INVENTION

The invention provides for a solid oral dosage form of methylphenidate or salts thereof. The dosage form comprises a matrix core comprising methylphenidate or salts thereof, a hydrophobic material in amount of up to about 10% w/w and a gel forming hydrophilic material in an amount of up to about 22% w/w along with other pharmaceutically acceptable excipients. The matrix core is coated with a modified release coating to further control the release of methylphenidate from the matrix core. An outer immediate release coating comprises methylphenidate or salts thereof.

The methylphenidate solid oral dosage forms according to the invention possess excellent batch-to-batch reproducibility in terms of release profile, even after exposing the formulations to accelerated stability conditions. The dosage form also exhibits prolonged duration of effect by extending release of methylphenidate from the formulation over a predetermined period of time.

The inventors have further observed that a typical composition of a methylphenidate solid oral dosage form in accordance with the invention rendered the dosage form to exhibit the aforesaid advantages. The inventors have also found that curing of the modified release coating is essentially not required during the manufacturing process due to the robustness of the matrix core and coating. As a result, relatively less optimization or control of the coating process is required during commercial scale manufacturing.

The term “methylphenidate”, as used herein, includes all optical isomers, racemic mixtures and the like of the compound and all pharmaceutically acceptable salts, amides, prodrugs and analogs thereof. The pharmaceutically active substance can be, for example, methylphenidate hydrochloride.

In one embodiment, the solid oral dosage form comprises:
(a) a matrix core which comprises methylphenidate or a salt thereof, at least one hydrophobic material, at least one gel forming hydrophilic material and one or more pharmaceutically acceptable excipients,
(b) a modified release coating surrounding the matrix core, wherein the modified release coating comprises at least one pH-independent polymer,
(c) optionally, a pH dependent release modifying coating surrounding the modified release coating,
(d) optionally, a seal coating surrounding the pH dependent release modifying coating, and
(e) a coating of methylphenidate or a salt thereof surrounding the modified release coating, pH dependent release modifying coating, or seal coating.

In an embodiment, the solid oral dosage form exhibits an in-vitro dissolution as follows when tested in standard dissolution conditions:
(a) not less than 10% methylphenidate is dissolved after 1 hour;
(b) not less than 40% methylphenidate is dissolved after 4 hours;
(c) not less than 80% methylphenidate is dissolved after 8 hours; and
(d) not less than 90% methylphenidate is dissolved after 12 hours.

The solid oral dosage form of the invention exhibits excellent storage stability when subjected to storage at 40° C. and 75% relative humidity for a period of 6 months. The dosage form retains at least 95% of the total potency of methylphenidate or salts thereof upon storage. Advantageously, no significant variation was observed between the release profile of the initial dosage form and the dosage form subjected to the stability testing.

The matrix core in the dosage form of the invention comprises methylphenidate or salts thereof, at least one hydrophobic material, at least one gel forming hydrophilic material and one or more pharmaceutically acceptable excipients.

Suitable hydrophobic material include, by way of example and without limitation, glyceride (e.g., glyceryl behenate, glyceryl trimyristate, glyceryl trilaurate, glyceryl tristearate, glyceryl monostearate, glyceryl palmitostearate, or glyceryl triacetate), hydrogenated castor oil, a hydrogenated vegetable oil, a wax or a wax-like substance (e.g., carnauba wax, cetyl esters wax, beeswax, castor wax, cationic emulsifying wax, cetrimide emulsifying wax, an emulsifying wax, microcrystalline wax, a nonionic wax, a nonionic emulsifying wax, paraffin, petroleum wax, petroleum ceresin wax, spermaceti wax, white wax, or yellow wax), a fat, an oil, a fatty acid or mixtures therefore. The hydrophobic material is preferably present in an amount up to about 10% w/w of the uncoated matrix core. In an embodiment, the hydrophobic material in the matrix core is not a polymeric material.

Suitable gel forming hydrophilic material include, by way of example and without limitation, polysaccharides, methyl cellulose, calcium carboxymethyl cellulose, hydroxypropyl methyl cellulose, hydroxypropyl cellulose, hydroxyethyl cellulose, nitrocellulose, carboxymethyl cellulose, cellulose ethers, poly(ethylene terphthalate), poly(vinyl isobutyl ether), polyurethane, polyethylene oxides (e.g. Polyox®, Union Carbide), methyl ethyl cellulose, ethylhydroxy ethylcellulose, cellulose acetate, ethylcellulose, cellulose butyrate, cellulose propionate, gelatin, collagen, starch, maltodextrin, pullulan, polyvinyl pyrrolidone, polyvinyl alcohol, polyvinyl acetate, glycerol fatty acid esters, polyacrylamide, polyacrylic acid, ammonio methacrylate copolymers such as Eudragit® RL or Eudragit® RS (e.g. Eudragit®, Rohm and Haas), other acrylic acid derivatives, sorbitan esters, polydimethyl siloxane, natural gums, lecithins, pectin, alginates, ammonia alginate, sodium, calcium, potassium alginates, propylene glycol alginate, agar, gums: arabic, karaya, locust bean, tragacanth, carrageenans, guar, xanthan, scleroglucan or mixtures thereof. The gel forming hydrophilic material is preferably present in an amount up to about 22% w/w of the uncoated matrix core.

Suitable additional pharmaceutically acceptable excipients that may be contained or present in the matrix core include, but are not limited to, binders, disintegrating agents, stabilizers, glidants, lubricants, granulating solvents and the like.

A modified release coating is applied over the matrix core to further retard the release of drug from the underlying core. Preferably, the modified release coating consists of at least one pH independent polymer. In a further embodiment, the modified release coating is devoid of a pH-dependent polymer or substance. In a further embodiment, the modified release coating is applied over the matrix core without any intermediate barrier coating.

Suitable pH independent polymers include, by way of example and without limitation, cellulose derivatives, chitosan derivatives, natural gums and polymethacrylates. Cellulose derivatives are selected from the group consisting of ethyl cellulose, hydroxypropyl cellulose, hydroxypropyl ethyl cellulose, hydroxyethyl cellulose, hydroxypropylmethyl cellulose, polyethylene oxides and hydroxymethyl cellulose, pH independent grades of methacrylic acid copolymers such as Eudragit™ RL and RS are pH-independent and the like.

In an embodiment, the modified release coating comprises at least one pH-independent polymer dissolved or dispersed in a hydro-alcoholic solvent mixture.

The dosage form of the invention further may comprise a pH dependent release modifying coating applied over the modified release coating. The pH dependent release modifying coating preferably comprises at least one pH dependent polymer, at least one hydrophilic pore forming agent and at least one plasticizer.

Suitable pH-dependent polymers include, by way of example and without limitation, methacrylic acid copolymers, cellulose acetate and its succinate and phthalate forms, styrol maleic acid co-polymers, polymethacrylic acid/acrylic acid copolymer, hydroxypropyl methyl cellulose phthalate, polyvinyl polymers, including polyvinyl acetate phthalate, hydroxyethyl ethyl cellulose phthalate, hydroxypropyl methyl cellulose acetate succinate, cellulose acetate tetrahydrophthalate, acrylic resin, timellitate, shellac, and the like. Preferably, the pH dependent polymer is a phthalate based polymer and not a methacrylic acid copolymer.

Suitable hydrophilic pore-forming agents include, by way of example and without limitation, a polymer (e.g., polyvinylpyrrolidone, a polyalkylene oxide, or a polyalkylene glycol), a cellulose or a cellulose ether (e.g., hypromellose, hypromellose phthalate, methyl cellulose, carboxymethyl cellulose sodium, hydroxyethyl cellulose, hydroxypropyl cellulose, carboxymethyl hydroxyethyl cellulose, ethyl hydroxyethyl cellulose, ethyl methyl cellulose, hydroxyethyl methyl cellulose, hydroxymethyl cellulose, or hydroxymethyl propyl cellulose), a protein, a protein derivative, a saccharide (e.g., pullulan, dextran, sucrose, glucose, fructose, mannitol, lactose, mannose, galactose, or sorbitol), a polysaccharide (e.g., polydextrose, guar gum, hydroxypropyl guar, alginate, xanthum gum or carboxymethyl hydroxypropyl guar), an alkali metal salt (e.g., lithium carbonate, sodium chloride, sodium bromide, potassium chloride, potassium sulfate, potassium phosphate, sodium acetate, or sodium citrate), and the like.

Suitable plasticizers include, by way of example and without limitation, triacetin, methyl abietate, acetyl tributyl citrate, acetyl triethyl citrate, diisooctyl adipate, amyl oleate, butyl ricinoleate, benzyl benzoate, butyl and glycol esters of fatty acids, butyl diglycol carbonate, butyl oleate, butyl stearate, di(.beta.-methoxyethyl) adipate, dibutyl sebacate, dibutyl tartrace, diisobutyl adipate, dihexyl adipate, triethylene glycol di(2-ethyl butyrate), polyethylene glycol di(2-ethyl hexoate), diethylene glycol monolaurate, monomeric polyethylene ester, hydrogenated methyl ester of rosin, methoxyethyl oleate, butoxyethyl stearate, butyl phthalyl butyl glycolate, glycerol tributyrate, and the like.

The dosage form further comprises an outer methylphenidate layer, coated either over the modified release coating, pH dependent release modifying coating or over the seal coating, if present in between. The outer coating of methylphenidate or a salt thereof preferably exhibits immediate release. In an embodiment, the outer coating comprises up to about 20% of methylphenidate or a salt thereof by total amount of methylphenidate in the dosage form.

Optionally, a seal coating is provided between the modified release coating and outer methylphenidate immediate release layer. The seal coat is preferably an inert or inactive composition, i.e., containing no active drug, and can comprise a polymeric compound, such as a cellulosic hydrogel, e.g., HPMC, as a film-former, a surfactant, such as polyethylene glycol, and a filler, such as talc. The composition of the seal coat can also comprise a pigment, such as titanium dioxide. The final dosage form can also include a seal coat surrounding the immediate release layer.

The invention further provides a method of manufacturing the solid oral dosage form of the invention comprising a matrix core, a modified release coating, a pH dependent release modifying coating, and a seal coating. The process comprises the steps of:
(a) forming a matrix core by compressing methylphenidate or a salt thereof, at least one hydrophobic material, and at least one gel forming hydrophilic material;
(b) applying the modified release coating over the matrix core;
(c) optionally, applying the pH dependent release modifying coating over the modified release coating;
(d) optionally, applying the seal coating over the modified release coating; and
(e) applying the coating of methylphenidate or a salt thereof over the modified release coating, pH dependent release modifying coating, or seal coating.

In an embodiment, the step of applying a modified release coating over the matrix core further comprises:
(a) dissolving or dispersing the pH-independent polymer in hydro-alcoholic solvent mixture to form a modified release coating composition; and
(b) applying the modified release coating composition over the matrix core.

The dosage form of the invention may be prepared in the form of a tablet, a capsule, or a capsule filled with granules, pellets, or minitablets.

In an embodiment, the methylphenidate modified release tablet comprises:
(a) a matrix core which comprises methylphenidate or a salt thereof, at least one hydrophobic material in an amount up to about 10% w/w of the uncoated matrix core, at least one gel forming hydrophilic material in an amount up to about 22% w/w of the uncoated matrix core, and one or more pharmaceutically acceptable excipients;
(b) a modified release coating surrounding the matrix core, wherein the modified release coating consists of at least one pH-independent polymer;
(c) optionally, a seal coating surrounding the pH dependent release modifying coating; and
(d) an outer immediate release coating comprising up to about 20% of methylphenidate or a salt thereof by total amount of methylphenidate in the dosage form.

The invention further provides a method for treating a patient having a condition amenable to treatment with methylphenidate, the method comprising administering to the patient the dosage form as substantially described herein.

In an embodiment, the invention relates to a method for treating Attention Deficit Disorder (ADD) or Attention Deficit/Hyperactivity Disorder (ADHD) in a patient in need thereof, said method comprising: providing a dosage form as substantially described herein and administering said dosage form to the patient.

### EXAMPLE 1

### Methylphenidate Hydrochloride Modified Release Tablet

TABLE 1

| Sr. | | Formulation (Qty/Batch in gm) | | | | |
|---|---|---|---|---|---|---|
| No. | Ingredients | 1 | 2 | 3 | 4 | 5 |

Matrix Core

| | | | | | | |
|---|---|---|---|---|---|---|
| 1 | Methylphenidate Hydrochloride | 368 | 352 | 368 | 360 | 352 |
| 2 | Hypromellose K100M | 240 | 240 | 200 | 184 | 176 |
| 3 | Colloidal silicon dioxide (Aerosil | 12 | 12 | 12 | 12 | 12 |
| | 200M) | | | | | |
| 4 | Lactose monohydrate (Modified | 1660 | 1676 | 1724 | 1828 | 1848 |
| | spray dried) | | | | | |
| 5 | Stearic acid (kolliwax S fine) | 120 | 120 | 96 | 0 | 0 |
| 6 | Magnesium stearate | 0 | 0 | 0 | 16 | 12 |

| | | | | | |
|---|---|---|---|---|---|
| TOTAL weight in mg | 300 | 300 | 300 | 300 | 300 |

Modified Release Coating

| | | | | | | |
|---|---|---|---|---|---|---|
| 7 | Ethylcellulose 7 cps | 58.7 | 56 | 56 | 56 | 56 |
| 8 | Hypromellose 15 cps | 38.2 | 37.3 | 37.3 | 37.3 | 37.3 |
| 9 | Triethyl citrate | 9.8 | 9.3 | 9.3 | 9.3 | 9.3 |
| 10 | Talc | 26.7 | 30.7 | 30.7 | 30.7 | 30.7 |
| 11 | Isopropyl alcohol | 1380 | 1380 | 1380 | 1380 | 1380 |
| 12 | Purified water | 153 | 153 | 153 | 153 | 153 |

Seal Coating

| | | | | | | |
|---|---|---|---|---|---|---|
| 13 | Opadry Clear YS-19025-A | 107 | 107 | 107 | 107 | 107 |
| 14 | Purified water | 960 | 960 | 960 | 960 | 960 |

Drug Coating

| | | | | | | |
|---|---|---|---|---|---|---|
| 15 | Methylphenidate Hydrochloride | 67.4 | 84.21 | 67.4 | 75.8 | 84.21 |
| 16 | Hypromellose E5 | 101 | 84.21 | 92.6 | 84.21 | 84.21 |
| 17 | Phosphoric acid | 0.34 | 0.34 | 0.34 | 0.34 | 0.34 |
| 18 | Purified water | 1520 | 1440 | 1440 | 1440 | 1520 |

Colour Coating

| | | | | | | |
|---|---|---|---|---|---|---|
| 19 | Opadry ™ II Beige 85F170014 | 124 | 124 | 124 | 124 | 124 |
| 20 | Purified water | 500 | 500 | 500 | 500 | 500 |

Procedure:

Core Tablets: Methylphenidate Hydrochloride, lactose monohydrate, hypromellose and colloidal silicon dioxide were sifted and mixed in a V-blender for 15 minutes. Stearic acid or magnesium stearate was sifted through #30 mesh and added to a V-blender and blended for 5 minutes. The final blend was compressed into tablets using a 0.3437″ round shaped tooling.

Modified Release Coating: Isopropyl alcohol and purified water were taken in a stainless steel container and mixed. Ethyl cellulose, hypromellose and triethyl citrate were added to the solvent mixture and mixed well to form a clear solution. Talc was added to the mixture and mixed well to form a uniform dispersion to form a coating solution which was then coated on to the core tablets in a perforated coating pan. (Process parameters: Exhaust temperature of 28-40° C. and Atomization pressure of approximately 10-20 psi). The coated tablets were dried in a coated pan at 40° C. for 30 minutes and collected.

Seal Coating: Opadry clear ready coating material was added into a clean stainless steel container containing purified water and mixed to form a clear coating solution. The coating solution was coated on to MR coated tablets in a perforated coating pan.

(Process parameters: Exhaust temperature of 35-45° C. and Atomization pressure of approximately 10-20 psi). The coated tablets were dried in the same coating pan at 40° C. for 30 minutes and collected.

Drug Coating: Hypromellose was added into a clean stainless steel container containing purified water and mixed to form a clear solution. Phosphoric acid was added to the solution to adjust the pH to 2.5-3.0. Methylphenidate hydrochloride was added to the solution under mixing to form a clear solution. The resulting solution was used to form a drug layer on the surface of the seal coated tablets in a perforated coating pan. (Process parameters: Exhaust temperature of 35-45° C. and Atomization pressure of approximately 10-20 psi). The coated tablets were dried in a coating pan at 40° C. for 30 minutes.

Colour Coating: Opadry™ beige ready coating material was added in a clean stainless steel container containing purified water and mixed well to form a uniform dispersion. The coating dispersion was used to coat the drug layered tablets in a perforated coating pan. (Process parameters: Exhaust temperature of 35-45° C. and Atomization pressure of approximately 10-20 psi). The coated tablets were dried in a coating pan at 40° C. for 30 minutes.

Dissolution Study

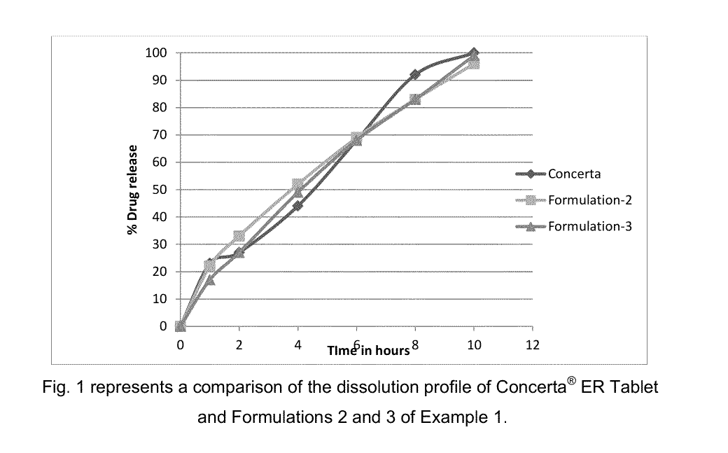

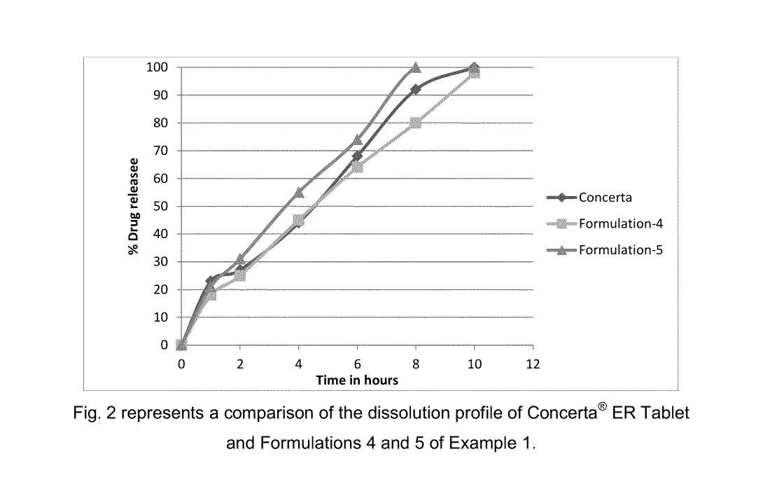

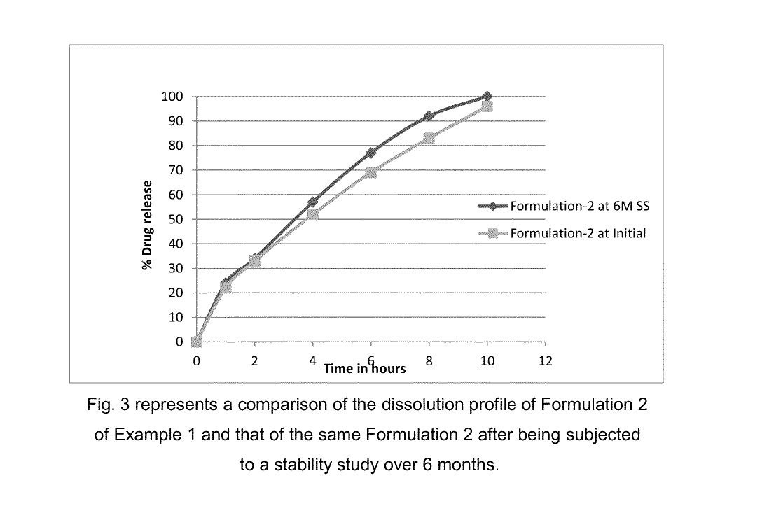

The modified release tablets of Example 1 were subjected to a dissolution study in standard conditions. Table 2 summarizes the dissolution profile of Formulations 1 to 5 of Example 1 and Formulation 2* relative to Concerts® ER Tablet. FIGS. 1 and 2 depict the equivalency between Formulations 5 and 6 and Concerts® ER Tablet. FIG. 3 depicts the equivalency between Formulation 2 and the same Formulation 2 (depicted as 2*) after being subjected to stability testing for 6 months.

TABLE 2

| Time | Concerta ® | Formulation | | | | | |
|---|---|---|---|---|---|---|---|
| (hr) | ER Tablet | 1 | 2 | 3 | 4 | 5 | 2* |
| 0 | 0 | 0 | 0 | 0 | 0 | 0 | 0 |
| 1 | 23 | 15 | 22 | 17 | 18 | 21 | 24 |
| 2 | 27 | 23 | 33 | 27 | 25 | 31 | 34 |
| 4 | 44 | 40 | 52 | 49 | 45 | 55 | 57 |
| 6 | 68 | 57 | 69 | 68 | 64 | 74 | 77 |
| 8 | 92 | 73 | 83 | 83 | 80 | 100 | 92 |
| 10 | 100 | 88 | 96 | 99 | 98 | 100 | 100 |

*% Dissolution of Formulation 2 after storing at 40° C. and 75% relative humidity for 6 months.

### COMPARATIVE EXAMPLE 1

### Methylphenidate Hydrochloride Modified Release Tablet

TABLE 3

| Sr. | | Formulation (Qty/Batch in mg) | | | | | |
|---|---|---|---|---|---|---|---|
| No. | Ingredient | 1 | 2 | 3 | 4 | 5 | 6 |

Intragranular Portion

| | | | | | | | |
|---|---|---|---|---|---|---|---|
| 1 | Methylphenidate | 54 | 54 | 54 | 54 | 54 | 54 |
| | Hydrochloride | | | | | | |
| 2 | Hypromellose | 125 | 125 | 112.5 | 125 | 70 | 80 |
| | (Methocel K100M | | | | | | |
| | Premium) | | | | | | |
| 3 | Hyprollose | 0 | 0 | 0 | 0 | 0 | 0 |
| | (Klucel HXF) | | | | | | |
| 4 | Hypromellose | 0 | 140 | 126 | 0 | 75 | 0 |
| | (Methocel K4M | | | | | | |
| | Premium) | | | | | | |
| 5 | Lactose | 40 | 114.5 | 102.7 | 99.5 | 60 | 30 |
| | Monohydrate | | | | | | |
| | (Modified | | | | | | |
| | spray dried | | | | | | |
| | lactose) | | | | | | |
| 6 | Stearic Acid | 7.5 | 7.5 | 6.75 | 7.5 | 5 | 5 |
| | (Kolliwax | | | | | | |
| | S fine) | | | | | | |
| 7 | Carbopol 971P | 99.5 | 0 | 0 | 50 | 0 | 50 |

Extragranular Portion

| | | | | | | | |
|---|---|---|---|---|---|---|---|
| 8 | Microcrystalline | 114 | 49 | 44.1 | 74 | 30 | 74.5 |
| | Cellulose | | | | | | |
| | (VIVAPUR ™ | | | | | | |
| | Type 102) | | | | | | |
| 9 | Colloidal Silicon | 2.5 | 2.5 | 2.25 | 2.5 | 1.5 | 1.5 |
| | Dioxide | | | | | | |
| 10 | Carbopol 71G | 0 | 0 | 0 | 0 | 0 | 0 |
| 11 | Stearic Acid | 7.5 | 7.5 | 6.75 | 7.5 | 5 | 7 |
| | (Kolliwax | | | | | | |
| | S fine) | | | | | | |

Total

Film Coating

| | | | | | | | |
|---|---|---|---|---|---|---|---|
| 12 | Opadry ™ | 14 | 15 | 14 | 12 | 10 | 10 |
| | II Beige | | | | | | |
| | 85F170014 | | | | | | |
| 13 | Purified water | Qs | Qs | Qs | Qs | Qs | Qs |

Procedure: The intragranular ingredients were sifted through a #16 mesh sieve and loaded into V-blender and mixed for 10 minutes. The blend was then compacted using a roller compactor with a screw speed of 9-25 rpm, a roller speed of 4-23 rpm, and a roller force of 6,000-30,000 lb. The compacts were milled through a #40G screen fitted with a Quadro comill. The milled granules then blended with extragranular ingredients. The combined blend was compressed into tablets using 0.4687 inch round standard cup toolings and with 0.3437 inch round standard cup toolings. The tablet parameters were as follows: tablet hardness of 10-20 kp, tablet friability of NMT 1.0% w/w. Finally, the compressed tablets were film coated with Opadry beige coloured coating material.

Dissolution Study

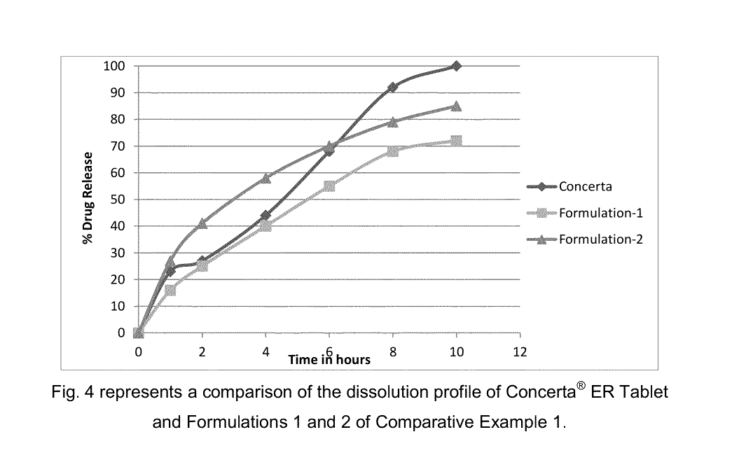

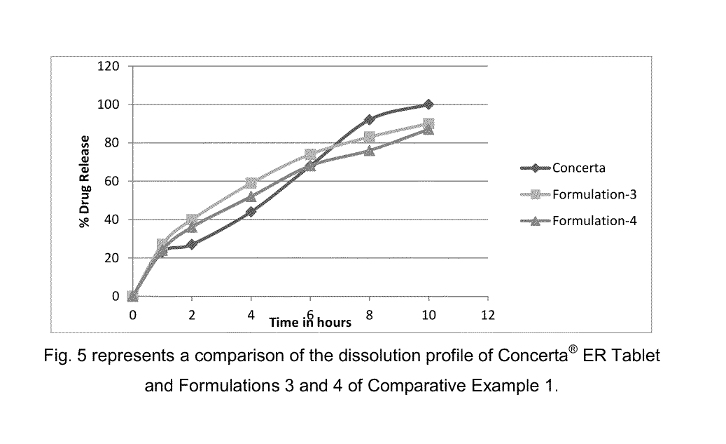

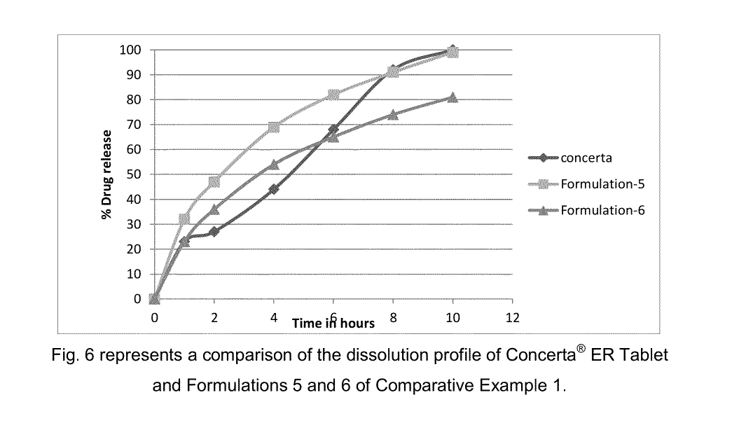

Comparative modified release tablets of Example 2 were subjected to dissolution study in standard conditions. Table 4 summarizes the dissolution profile of Formulations 1 to 5 and Formulation 2* relative to Concerts® ER Tablet. FIGS. 4, 5 and 6 compare the dissolution profile between Formulations 5 and 6 and Concerts® ER Tablet.

TABLE 4

| | | |
|---|---|---|
| Time | Concerta ® | Formulation |

| (hr) | ER Tablet | 1 | 2 | 3 | 4 | 5 | 2* |
|---|---|---|---|---|---|---|---|
| 0 | 0 | 0 | 0 | 0 | 0 | 0 | 0 |
| 1 | 23 | 16 | 27 | 27 | 24 | 32 | 23 |
| 2 | 27 | 25 | 41 | 40 | 36 | 47 | 36 |
| 4 | 44 | 40 | 58 | 59 | 52 | 69 | 54 |
| 6 | 68 | 55 | 70 | 74 | 68 | 82 | 65 |
| 8 | 92 | 68 | 79 | 83 | 76 | 91 | 74 |
| 10 | 100 | 72 | 85 | 90 | 87 | 99 | 81 |

1. A solid oral dosage form comprising:
(a) a single, compressed matrix core comprising methylphenidate or a salt thereof, at least one hydrophobic material, at least one gel forming hydrophilic material and one or more pharmaceutically acceptable excipients;
(b) a modified release coating surrounding the matrix core, the modified release coating comprising at least one pH-independent polymer;
(c) optionally, a pH dependent release modifying coating surrounding the modified release coating;
(d) optionally, a seal coating surrounding the pH dependent release modifying coating; and
(e) an outer coating of methylphenidate or a salt thereof surrounding the modified release coating, pH dependent release modifying coating, or seal coating, wherein the outer coating comprises less than 20% by weight of the methylphenidate or a salt thereof present in the dosage form;
wherein the matrix core comprises up to about 10% w/w of the at least one hydrophobic material and up to about 22% w/w of the at least one gel forming hydrophilic material by total weight of the uncoated matrix core.

2. The dosage form of claim 1, wherein the modified release coating comprises at least one pH independent polymer, wherein the pH independent polymer is applied to surround the matrix core from a solution of the pH independent polymer dissolved or dispersed in a hydro-alcoholic solvent mixture.

3. The dosage form of claim 1, wherein the modified release coating is devoid of a pH dependent polymer or substance.

4. The dosage form of claim 1, wherein the outer coating of methylphenidate or a salt thereof exhibits immediate release.

5. (canceled)

6. The dosage form of claim 1, wherein the hydrophobic material is not a polymeric material.

7. The dosage form of claim 1, wherein the modified release coating is applied over the matrix core without any intermediate barrier coating.

8. The dosage form of claim 1, wherein the hydrophobic material is selected from the group consisting of glyceryl behenate, glyceryl trimyristate, glyceryl trilaurate, glyceryl tristearate, glyceryl monostearate, glyceryl palmitostearate, glyceryl triacetate, hydrogenated castor oil, a hydrogenated vegetable oil, a wax carnauba wax, cetyl esters wax, beeswax, castor wax, cationic emulsifying wax, cetrimide emulsifying wax, an emulsifying wax, microcrystalline wax, a nonionic wax, a nonionic emulsifying wax, paraffin, petroleum wax, petroleum ceresin wax, spermaceti wax, white wax, a fat, an oil, and a fatty acid.

9. The dosage form of claim 1, wherein the gel forming hydrophilic material is selected from the group consisting of polysaccharides, methyl cellulose, calcium carboxymethyl cellulose, hydroxypropyl methyl cellulose, hydroxypropyl cellulose, hydroxyethyl cellulose, nitrocellulose, carboxymethyl cellulose, cellulose ethers, poly(ethylene terphthalate), poly(vinyl isobutyl ether), polyurethane, polyethylene oxides, methyl ethyl cellulose, ethylhydroxy ethylcellulose, cellulose acetate, ethylcellulose, cellulose butyrate, cellulose propionate, gelatin, collagen, starch, maltodextrin, pullulan, polyvinyl pyrrolidone, polyvinyl alcohol, polyvinyl acetate, glycerol fatty acid esters, polyacrylamide, polyacrylic acid, ammonio methacrylate copolymers, natural gums, lecithins, pectin, alginates, ammonia alginate, sodium, calcium, potassium alginates, propylene glycol alginate, agar, gum arabic, gum karaya, locust bean gum, tragacanth, carrageenans, guar, xanthan, and scleroglucan.

10. The dosage form of claim 1, wherein the pH dependent release modifying coating comprises at least one pH-dependent polymer, at least one hydrophilic pore forming agent and at least one plasticizer.

11. The dosage form of claim 10, wherein the pH-dependent polymer is selected from the group consisting of shellac, cellulose acetate phthalate, polyvinyl acetate phthalate, hydroxypropyl methylcellulose phthalate, a pH dependent methacrylic acid ester copolymer, and zein.

12. The dosage form of claim 1, wherein the dosage form provides an in-vitro dissolution as follows:
(a) not less than 10% methylphenidate is dissolved after 1 hour;
(b) not less than 40% methylphenidate is dissolved after 4 hours;
(c) not less than 80% methylphenidate is dissolved after 8 hours; and
(d) not less than 90% methylphenidate is dissolved after 12 hours.

13. The dosage form of claim 1, wherein the release profile of said dosage form after subjecting the dosage form to storage at 40° C. and 75% relative humidity for a period of 6 months is the following:
(a) not less than 10% methylphenidate is dissolved after 1 hour;
(b) not less than 40% methylphenidate is dissolved after 4 hours;
(c) not less than 80% methylphenidate is dissolved after 8 hours; and
(d) not less than 90% methylphenidate is dissolved after 12 hours.

14. A method of manufacturing the dosage form of claim 1, the method comprises the steps of:
(a) preparing the matrix core by compressing methylphenidate or a salt thereof, at least one hydrophobic material, and at least one gel forming hydrophilic material;
(b) applying the modified release coating over the matrix core;
(c) optionally, applying the pH dependent release modifying coating over the modified release coating;
(d) optionally, applying the seal coating over the modified release coating; and
(e) applying the coating of methylphenidate or a salt thereof over the modified release coating, pH dependent release modifying coating, or seal coating.

15. The method of claim 14, wherein step (b) of the manufacturing method further comprises:
(a) dissolving or dispersing the pH-independent polymer in a hydro-alcoholic solvent mixture to form a modified release coating composition; and
(b) applying the modified release coating composition over the matrix core.

16. A method for treating a patient having Attention Deficit Disorder or Attention Deficit/Hyperactivity Disorder, the method comprising administering to the patient the dosage form according to claim 1.

17. A method for treating Attention Deficit Disorder or Attention Deficit/Hyperactivity Disorder in a patient in need thereof, said method comprising providing the dosage form of claim 1 and administering said dosage form to the patient.

18. A methylphenidate modified release tablet comprising:
(a) a single matrix core which comprises methylphenidate or a salt thereof, at least one hydrophobic material in an amount up to about 10% w/w of the uncoated matrix core, at least one gel forming hydrophilic material in an amount up to about 22% w/w of the uncoated matrix core, and one or more pharmaceutically acceptable excipients;
(b) a modified release coating surrounding the matrix core, wherein the modified release coating consists of at least one pH-independent polymer;
(c) optionally, a seal coating surrounding the pH-independent modified release coating; and
(d) an immediate release coating surrounding the modified release coating and comprising up to 20% of methylphenidate or a salt thereof by total amount of methylphenidate in the dosage form.

19. The dosage form of claim 1, wherein the outer coating of methylphenidate comprises between 15.4% and 19.3% by weight of the methylphenidate or a salt thereof present in the dosage form.

20. The methylphenidate modified release tablet of claim 18, wherein the immediate release comprises between 15.4% and 19.3% by weight of the methylphenidate or a salt thereof present in the dosage form.
